(19) United States
(12) Patent Application Publication
(10) Pub. No.: US 2016/0015324 A1
(43) Pub. Date: Jan. 21, 2016

(54) System, Method, and Apparatus for Measuring Pulmonary Data

(71) Applicant: Dean Du Bois, Clearwater, FL (US)

(72) Inventor: Dean Du Bois, Clearwater, FL (US)

(21) Appl. No.: 14/333,585

(22) Filed: Jul. 17, 2014

(51) Int. Cl. A61B 5/00; A61B 5/087; A61B 5/097

(52) U.S. Cl. CPC A61B 5/6898; A61B 5/097; A61B 5/087; A61B 5/7278; A61B 5/0004; A61B 5/486; A61B 5/7405; A61B 5/742

(57) ABSTRACT

A system for gathering pulmonary data includes a smartphone and a back cover for the smartphone. The smartphone has a processor, a display, and a pressure sensor. The pressure sensor is operatively coupled to the processor (e.g. electrically coupled) and the display is also operatively coupled to the processor. The back cover attaches to the back of the smartphone and has an interface for accepting a breath from a user and routing the breath through at least one channel such that the breath is fluidly interfaced between the interface for accepting the breath and the pressure sensor. The application gathers samples of the breath to calculate exhalation volume and pressures and, in some examples, generates and displays a spirogram from the data.

### FIELD

This invention relates to the field of health monitoring and more particularly to a system for performing measurements of respiration.

### BACKGROUND

In some countries, the cellular phone has become ubiquitous, with almost every adult and many children possessing a cellular phone and, most likely, a smartphone. The primary purpose of any phone is to make voice calls, but newer phones are moving to provide many other features, almost to a point where making voice calls becomes secondary. Cellular phones include a processor, a display, and input/output devices (microphone, speaker, touch-screen, digital image sensors). Many such phones have displaced cameras for taking pictures by having high-quality digital image sensors, have displaced personal computers for general computing (e.g., searching, messaging), have displaced stand-alone Global Positioning devices, etc. Today, cellular phones are changing entire corporations, just as digital cameras changed the film industry. Smartphones have reduced sales of digital cameras, stand-alone GPS navigation systems, pagers, etc.

Recently, other input devices have been included on some cellular phones to further improve the usefulness and features of such phones, in particular, for health and fitness. For example, at least one manufacturer has included a pulse detector integrated into a back surface of a smartphone, using a light emitting diode to shine a light through a user's finger and measuring the amount of light reflected back to determine blood flow, then, this manufacture provides software that analyzes the reflected light to calculate and display the user's pulse rate, etc.

As early as 2013, manufacturers of smartphones have included sensors such as accelerometers to measure acceleration and rotational forces, temperature sensors, air pressure sensors, illumination sensors, humidity sensors, orientation sensors, and magnetometer sensors (e.g. compass). Many of these sensors are accessed and used by the smartphone systems to provide user interface features such as detecting when the smartphone is shaken to randomize a music play list or for detecting when the smartphone is currently within a user's pocket or handbag to prevent “pocket calls.” Some sensors are often used to protect the smartphone from, for example, theft, overheating, etc. Some of these sensors are also used to provide information to the user such as which way is north, or how many steps the user has taken during a period of time as feedback regarding exercise regimes.

Most sensors are internal to the smartphone and primarily intended for purposes decided by the smartphone manufacturer. Typically, these sensors are mounted on a circuit board within the smartphone and housed somewhere between the case, display, and back cover of the smartphone. For example, a Global Positioning Service receiver within the enclosure of the smartphone is intended to provide the smartphone with location information of the smartphone. This, coupled with another internal sensor, a magnetometer, helps the smartphone determine location, orientation, and direction of travel.

Recently, at least one smartphone manufacture has included a pressure sensor on several models of smartphones. This internal pressure sensor is intended to be used by the Global Positioning System to determine the altitude of the smartphone while the smartphone locks onto satellite signals, providing quicker locking (less “searching for satellite” messages). It is also anticipated that, in the future, such pressure sensors will assist with indoor navigation. The pressure sensor is buried within the enclosure of the cellular phones and hidden behind the back cover. To date, this pressure sensor has not been used to provide any health-related features.

What is needed is a system that communicates with the pressure sensor of a cellular phone and providing respiratory-related data.

### SUMMARY

In one embodiment, a system for gathering pulmonary data is disclosed including a smartphone. The smartphone has a processor, a display, and a pressure sensor. The pressure sensor is operatively coupled to the processor (e.g. electrically coupled) and the display is also operatively coupled to the processor. The system includes a back cover for attaching to the back of the smartphone. The back cover has an interface for accepting a breath at least one channel through which the breath is fluidly interfaced between the interface for accepting the breath and the pressure sensor.

In another embodiment, a method of gathering pulmonary data is disclosed including affixing a custom back cover to a smartphone. The custom back cover has an interface for accepting a breath from a user and has at least one channel through which the breath is fluidly interfaced between the interface for accepting the breath and a pressure sensor of the smartphone. The pressure sensor is operatively coupled to a processor of the smartphone. The method includes starting an application running on the processor of the smartphone and the application displaying instructions on a display of the smartphone (e.g., “inhale deeply, engage mouth with the interface for accepting the breath, and exhale as hard and quickly as possible”). The display is also operatively coupled to the processor. A user following the instructions exhales into the interface for accepting a breath and the application gathers exhalation data from the pressure sensor while the user is exhaling.

In another embodiment, an apparatus for measuring pulmonary capabilities is disclosed including a cover of size and form for interfacing with the back of a smartphone and a pulmonary interface on (e.g. mounted to, affixed to, part of) the cover. The pulmonary interface has an interface for accepting a breath and at least one channel through which a breath is fluidly interfaced between the interface for accepting a breath and a location of where the pressure sensor of the smartphone is located.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of the internal components of one typical smartphone of the prior art.

FIG. 2 illustrates a perspective view of the inside area of a pulmonary interface.

FIG. 3 illustrates a perspective view of the outer area of the pulmonary interface.

FIG. 4 illustrates an exploded view of the pulmonary interface.

FIG. 5 illustrates a perspective view of the pulmonary interface showing one example of an adjustable resistance device.

FIG. 6 illustrates a cut-away view of the pulmonary interface showing air flow.

FIG. 7 illustrates an exploded view of a second exemplary pulmonary interface system with multiple interface tubes.

FIG. 7A illustrates an alternate connection system.

FIG. 8 illustrates a schematic view of a typical smartphone system.

FIGS. 9A, 9B, and 9C illustrate a schematic view of an exemplary pulmonary software flowchart.

FIG. 10 illustrates a schematic view of a second exemplary pulmonary software flowchart.

FIG. 11 illustrates a schematic view of a third exemplary pulmonary software flowchart.

FIG. 12 illustrates a schematic view of an exemplary pulmonary data entry user interface.

FIG. 13 illustrates a schematic view of a second exemplary pulmonary data entry user interface.

FIG. 14 illustrates a schematic view of a third exemplary pulmonary data entry user interface.

FIG. 15 illustrates a plan view of a fourth exemplary pulmonary user interface.

FIG. 16 illustrates a plan view of a fifth exemplary pulmonary user interface.

FIG. 17 illustrates a plan view of a sixth exemplary pulmonary user interface.

FIG. 18 illustrates a schematic view of a seventh exemplary pulmonary data entry user interface showing an output graphic.

FIG. 19 illustrates a schematic view of flow in an exemplary pulmonary data system.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout this document, the term smartphone is used, in general, to describe any cellular phone, including cellular phones with keyboards, cellular phones with touch screens, cellular phones with voice command, etc. The disclosure is not dependent upon any particular type, manufacturer or variety of cellular phone (smartphone), the only requirement being some form of pressure transducer being available within the cellular phone (smartphone).

Pulmonary function testing is a tool for evaluating the respiratory system. During pulmonary function testing, exhalation values are compared to values derived from a sample study to determine/predict lung diseases such as fibrosis, etc. In this, a device/machine measures exhalation pressure and charts a spirogram. During this test, typically, a seated patient inhales maximally from tidal respiration to total lung capacity and then rapidly exhales to their fullest extent until no further volume is exhaled. A spirogram is a graphical representation of respiratory movements during this test, including any coughing that occurs during inhalation and exhalation.

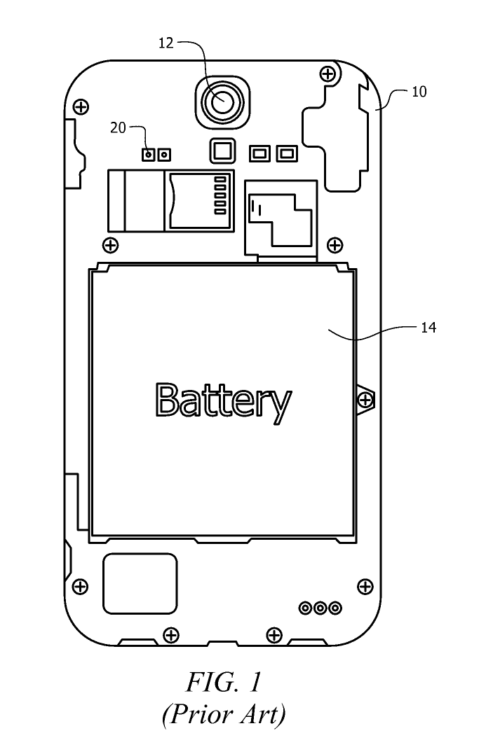

Referring to FIG. 1, a schematic view of the internal components of one typical smartphone 10 of the prior art is shown. The smartphone 10 is an example of, per se, a typical smartphone 10 having the back cover removed, exposing internal components and battery 14. In this exemplary smartphone 10, the battery 14 is somewhat centrally located and the image sensor 12 and other internal components are visible. Recently, some smartphones 10 include a pressure transducer 20 for measurement of, for example, atmospheric pressure. Software running on such smartphones 10 has access to data from this pressure transducer 20. Having such data, the software running on this smartphone 10 is able to calculate various environmental parameters related to the location of the smartphone 10, such as, altitude, changes in altitude, Y-axis directional movement of the smartphone 10, local barometric pressure (e.g. for predicting weather changes), etc.

The pressure sensor 20 used in many smartphones 10 is a micro-electromechanical system (MEMS) pressure sensors. The incentive to include such a pressure sensor 20 within a smartphone 10 is driven by improving the speed at which the Global Positioning System (GPS) of the smartphone 10 will lock, wherein the GPS chipset is better able to lock on to a satellite signal and calculate positions more quickly by using the pressure sensor 20 to determine the smartphone's 10 altitude. As an example, one such pressure sensor 20 is capable of measuring pressures of from 260 to 1260 mbar with a resolution of 0.020 mbar.

For the perceived uses of this pressure sensor 20, the typical smartphones 10 include the pressure sensor 20 at a location internal to the smartphones 10, typically behind the back cover of the smartphone 10. It is anticipated that air pressure from the outside of the enclosure encapsulating the smartphone 10 equalizes with pressure within the enclosure of the smartphone 10 through tiny holes in the enclosure (e.g. speaker and microphone portals) and/or leakage between the enclosure and the back cover of the smartphone 10. The prior art does not provide any external access to the pressure sensors 20 through the existing back covers of current smartphones 10.

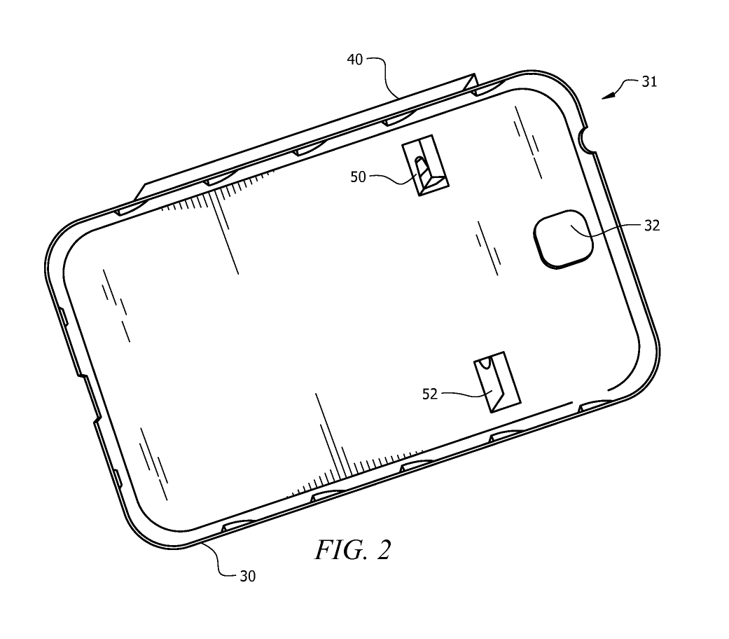

Referring to FIG. 2 illustrates a perspective view of the inside area of a custom cover 30 with an exemplary pulmonary interface 31 is shown. A modified or custom back cover 30 includes one or more pulmonary ports 50/52 through which pulmonary air flow is directed to/from the location of the pressure sensor 20 of a smartphone 10. Although two pulmonary ports 50/52 are shown, any number and/or location of pulmonary ports 50/52 is anticipated, correlated to any number and/or location of pressure sensors 20 within a target range of smartphones 10 that include such pressure sensors 20. In other words, it is anticipated that, for a specific smartphone 10 architecture (e.g. enclosure size, shape, and back cover), a single modified back cover 30 is provisioned having properly located pulmonary ports 50/52 to interface with one or more locations of pressure sensors 20 within a range of smartphones 10 within this architecture of smartphones 10. Being such, if a given architecture of smartphones 10 (e.g. case shape, size, back cover interface, etc.) has two models of smartphones 10, model A and model B, and each has a pressure sensor 20, but the location of the pressure sensor 20 is different between both model A and model B, then the same modified back cover 30 is used for both model A and model B. When this modified back cover 30 is installed on a Model A smartphone 10, pulmonary air flow is directed from the first pulmonary port 50 onto the pressure sensor 20 of the Model A smartphone 10. When this modified back cover 30 is installed on a Model B smartphone 10, pulmonary air flow is directed from the second pulmonary port 52 onto the pressure sensor 20 of the Model B smartphone 10.

As previously stated, in some embodiments, there is only one pulmonary port 50. Also, for completeness, a portal or window 32 is provided for the image sensor 12 of the smartphone 10 and any number, size, and type of back cover feature such as the portal 32 is anticipated as is found in the original back cover (not shown) of the smartphone 10.

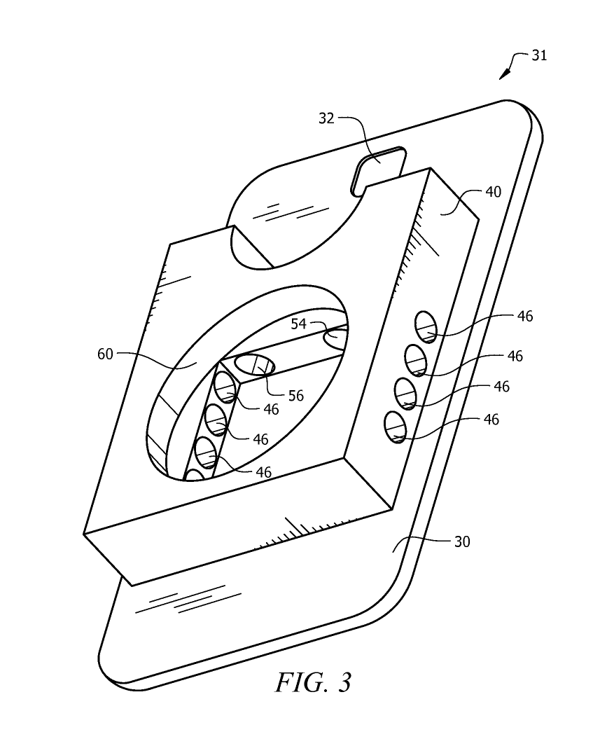

Referring to FIGS. 3 through 6, perspective views of the exemplary pulmonary interface 31 are shown. The pulmonary interface 31 provides both passage of pulmonary air flow from a patient (not shown) to the pressure sensor 20 of a smartphone 10 and a certain back-flow resistance to the pulmonary air flow. Back-flow resistance is used to meter the inhalation and/or exhalation pressure and volume of the patient as the patient inhales and exhales through the pulmonary interface 31.

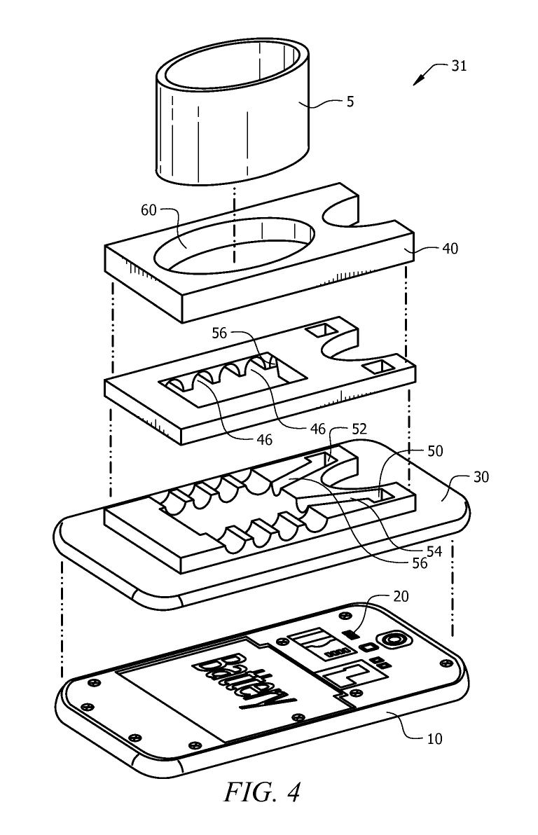

In FIG. 3, a patient interface port 60 is shown on an enclosure 40 of the exemplary pulmonary interface 31 (e.g. affixed to the custom cover 30 or formed as part of the custom cover 30). The patient interface port 60 (or interface for accepting a breath 60) interfaces with the patient (not shown) through any way known in the industry including: a disposable sampling tube 5 (see FIG. 4), through a flexible tube (not shown), through a tracheotomy tube (not shown), etc. As the pulmonary air flows in/out of the patient interface port 60, some of the pulmonary air flows in/out of exit vents 46, providing a certain amount of back-flow resistance. Some of the pulmonary air flows in/out of ducts 54/56, which are in fluid communication with the one or more pulmonary ports 50/52, providing some amount of the pulmonary air flow directed to the pressure sensor 20 of the smartphone 10, when the modified back cover 30 is installed on the smartphone 10.

In some embodiments of the modified back cover 30, a single, non-adjustable back-flow resistance is provided by, for example, having a certain number and size of exit vents 46, such as a single exit vent 46 or two exit vents 46.

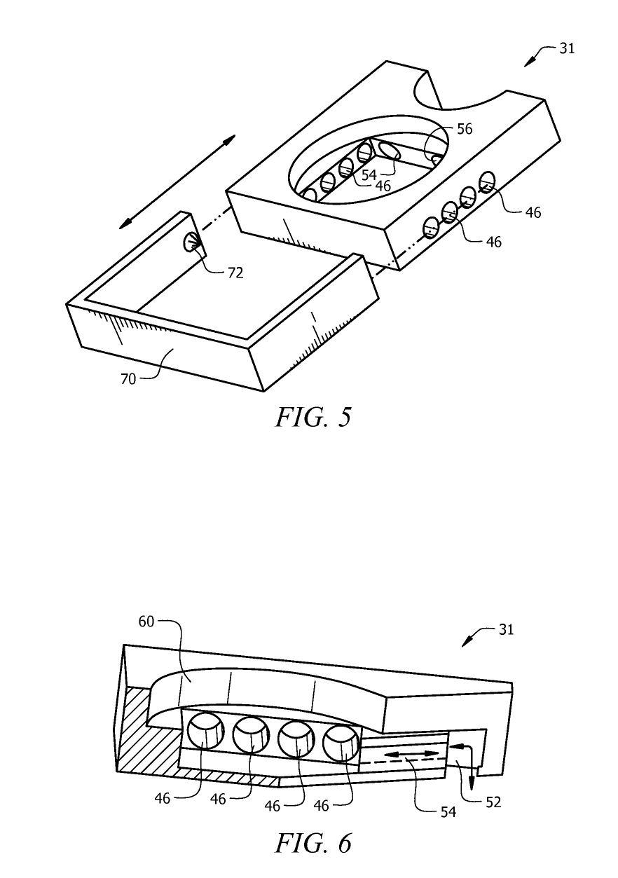

Different patients possess different basic abilities to inhale and exhale, including pressure and volume expulsion. For example, one might expect that a patent having emphysema or a patent with part of their lung removed to have less volume and pressure than a healthy patient. To provide accommodation for a range of patient capabilities, in some embodiments, an adjustable back-flow resistance mechanism is provided, adjusting the overall volume of the exit vents 46 by, for example, occluding some portion to the exit vents 46 through a dial mechanism, slider, or other. Although there are many ways to adjust the occlusion of the exit vents 46, one such adjustment mechanism 70/72 is shown in FIG. 5. In this, a sliding cover 70 occludes one or more of the exit vents 46 and is held within one pair of the exit vents 46 by resilient force of the sliding cover 70 and a protrusion 72 that fits within one pair of the exit vents 46. In use, for a healthy patient with no lung issues, the sliding cover 70, for example, is removed; while for a very ill patient with extremely limited lung capacity, most of the exit vents 46 are covered. Although there is no absolute restriction, it is preferred that at least one exit vent 46 be open during operation, though it is anticipated that all exit vents 46 be occluded when not in use to reduce dirt and dust penetration into the phone.

The number of occluded exit vents 46 effect the sensitivity and maximum range of breath velocity that can be measured. Tuning the number of occluded exit vents 46 for a given patient provides maximum accuracy. More exit vents 46 are occluded or closed to compensate for the severity of the lung deficiency and/or to increase the sensitivity of the apparatus for measuring pulmonary data.

FIG. 6 shows the pulmonary air flow from the patient interface port 60 through one of the ducts 56 and in/out of one of the pulmonary ports 52 which is in fluid communication with the pressure sensor 20 of a smartphone 10 on which the modified back cover 30 is installed.

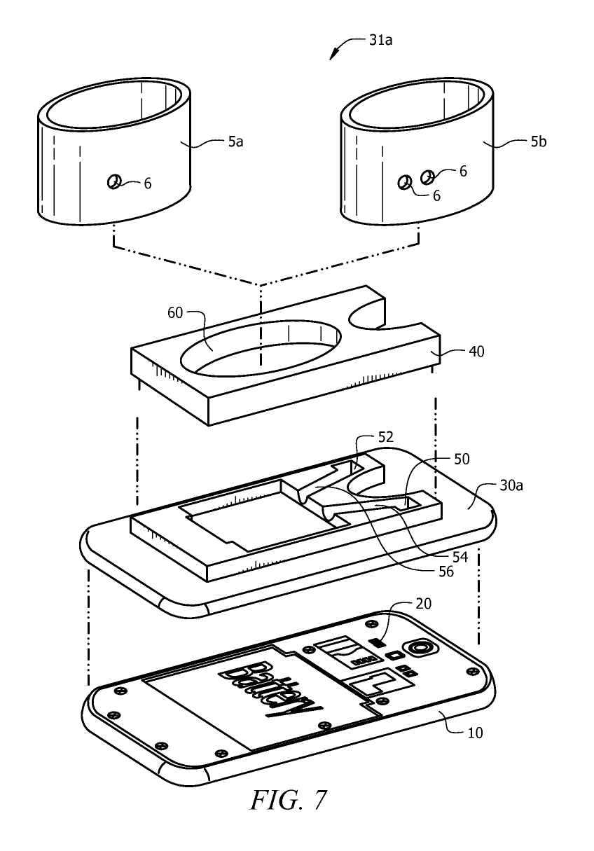

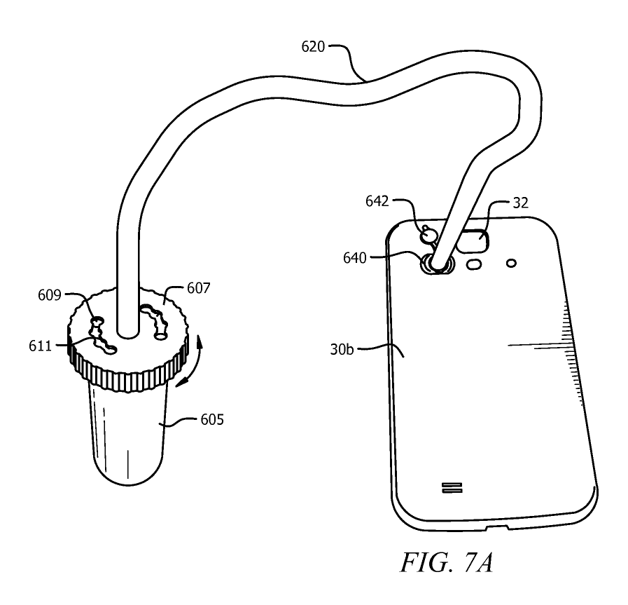

Referring to FIG. 7, an exploded view of a second exemplary pulmonary interface system 31a with alternate multiple sampling tubes 5a/5b is shown. In general, most users wish to have a smartphone 10 that is as thin and light-weight as practical. Most adjustable back-flow resistance mechanism require some type of lever, slider, knob, etc., that is adjustable by, for example, a user's fingers. Such mechanisms consume space, often require additional thickness, and add weight. In an alternate embodiment, the modified back cover 30A is without any adjustable back-flow resistance mechanism and, instead, a variety of sampling tubes 5a/5b (disposable or not) are available having different volumes of exit vents 6. By selecting one of the available sampling tubes 5a/5b, the patient is provided with a specific amount of back-flow resistance determined by the overall total volume of the exit vents 6 (if present). For example, a sampling tube 5a with one vent 6 is prescribed for a patient with less healthy lungs, and a sampling tube 5b with two vents 6 is prescribed for a patient with healthier lungs. In some embodiments, multiple vents 6 are provided, while in other embodiments, the size (volume) of the vents 6 is varied (e.g. a fixed number of vents 6 of different sizes). Although three specific sampling tubes 5/5a/5b have been shown, any number, configuration, size, and back-flow resistance characteristics are anticipated. In some embodiments, multiple vents 6 are located on various sides of the sampling tubes 5a/5b to reduce the possibility of occlusion of all of the vents 6 by, for example, the patient's fingers or lips. In some embodiments, each sampling tube 5a/5b has the same number of vents 6 (e.g. two vents 6 spaced 180 degrees apart) and the vents 6 of a first sample tube 5a have a first cross-sectional area and the vents 6 of a second sample tube 5b have a second cross-sectional area and the first cross-sectional area is less than the second cross-sectional area.

The more open vents 6, the larger of a velocity can be detected. Deficient lungs and lungs of children typically require fewer (open) vents 6 than healthy adults.

In some embodiments, the sampling tube 5/5a/5b is long enough and/or shaped to wrap around to the front area of the smartphone 10, allowing the user/patient view of the display 86 while performing the testing. In one such example, a flexible tube is interfaced to the patient interface port 60 and a distal end of the flexible tube has a mouthpiece and/or disposable sampling tube ends. In such, it is anticipated that the patient interface port 60 is sized to accept both the sampling tubes 5/5a/5b or a flexible sampling tube (not shown) of a lesser diameter, etc.

Referring to FIG. 7A, an alternate connection system with a flexible, elongated sampling tube extension 620 is shown. In this, the modified back cover 30b has a port 640 that, when not in use, has a cover 642 to reduce introduction of dust, liquids, and other debris ito the smartphone 10. When in use, an elongated sampling tube extension 620 fluidly connects the sampling tube 605 to the port 640, which is located above the pressure sensor 20. One end of the elongated sampling tube extension 620 interfaces with the port 640 and the other end of the elongated sampling tube extension 620 interfaces with a sampling tube 605, through for example, an adjustable baffle 607/609/611. The elongated sampling tube extension 620 receives breath pressure samples from the sampling tube 605 which, in use, is held in the lips of the user. Adjustments are made to the adjustable baffle 607/609/611 to adjust the amount of air from each breath that is allowed to escape vs. the amount of air from each breath that is channeled into the elongated sampling tube extension 620. For example, by rotating the control top 607, zero, one, or several holes or openings 609 are exposed through the orifice 611. For a healthy patient, more openings 609 are exposed while for an patient with minimal lung capacity, less openings 609 are exposed, possibly no openings 609 are exposed for a patient with very poor lung capacity.

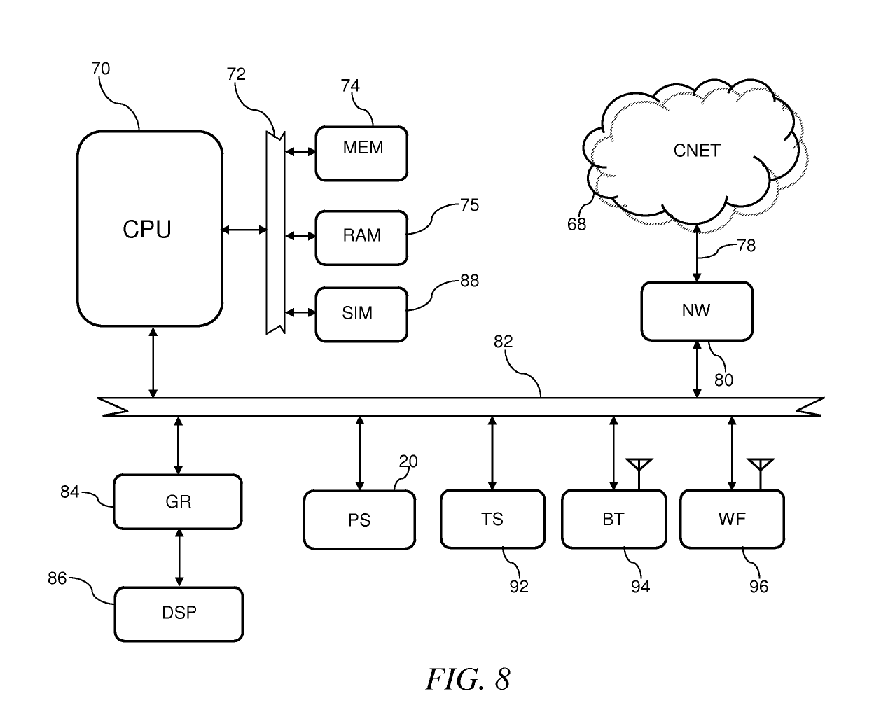

The elongated sampling tube extension 620 allows for the user/patient to perform the pulmonary tests while receiving instructions and feedback on the display 86 (see FIG. 8) of the smartphone 10. For example, a graphical representation of the current breath is anticipated to provide feedback to the user/patient as to how well they are doing in the test, perhaps color changes where green indicates the user/patient is doing well (e.g., compared to a prior test) and red indicates the user/patient is not doing as well, etc.

Referring to FIG. 8, a schematic view of a typical smartphone 10 is shown. The example smartphone 10 represents a typical phone system used with the mechanisms 31/31a for measuring pulmonary air flows. This exemplary smartphone 10 is shown in its simplest form. Different architectures are known that accomplish similar results in a similar fashion and the present invention is not limited in any way to any particular smartphone 10 system architecture or implementation. In this exemplary smartphone 10, a processor 70 executes or runs programs in a random access memory 75. The programs are generally stored within a persistent memory 74 and loaded into the random access memory 75 when needed. Also accessible by the processor 70 is a SIM (subscriber information module) card 88 having a subscriber identification and often persistent storage. The processor 70 is any processor, typically a processor designed for phones. The persistent memory 74, random access memory 75, and SIM card are connected to the processor by, for example, a memory bus 72. The random access memory 75 is any memory 75 suitable for connection and operation with the selected processor 70, such as SRAM, DRAM, SDRAM, RDRAM, DDR, DDR-2, etc. The persistent memory 74 is any type, configuration, capacity of memory 74 suitable for persistently storing data, for example, flash memory, read only memory, battery-backed memory, magnetic memory, etc. In some exemplary smartphones 10, the persistent memory 74 is removable, in the form of a memory card of appropriate format such as SD (secure digital) cards, micro SD cards, compact flash, etc.

Also connected to the processor 70 is a system bus 82 for connecting to peripheral subsystems such as a cellular network interface 80, sensors such as a pressure sensor 20, a graphics adapter 84 and a touch screen interface 92. The graphics adapter 84 receives commands from the processor 70 and controls what is depicted on a display image on the display 86.

In general, some portion of the memory 74 and/or the SIM 88 is used to store programs, executable code, phone numbers, contacts, and data such as user health data in a persistent manner. In some embodiments, other data is stored in the memory 74 such as audio files, video files, text messages, etc.

The peripherals and sensors shown are examples and other devices are known in the industry such as Global Positioning Subsystems, speakers, microphones, USB interfaces, Bluetooth transceivers 94, Wi-Fi transceivers 96, image sensors, temperature sensors, etc., the likes of which are not shown for brevity and clarity reasons.

The network interface 80 connects the smartphone 10 to the cellular network 68 through any cellular band and cellular protocol such as GSM, TDMA, LTE, etc. There is no limitation on the type of cellular connection used. The network interface 80 provides voice call, data, and messaging services to the smartphone 10 through the cellular network.

For local communications, many smartphones 10 include a Bluetooth radio transceiver 94, a Wi-Fi radio transceiver 96, or both. Such features of smartphones 10 provide data communications between the smartphones 10 and other computers such as a personal computer of the patient/user (not shown).

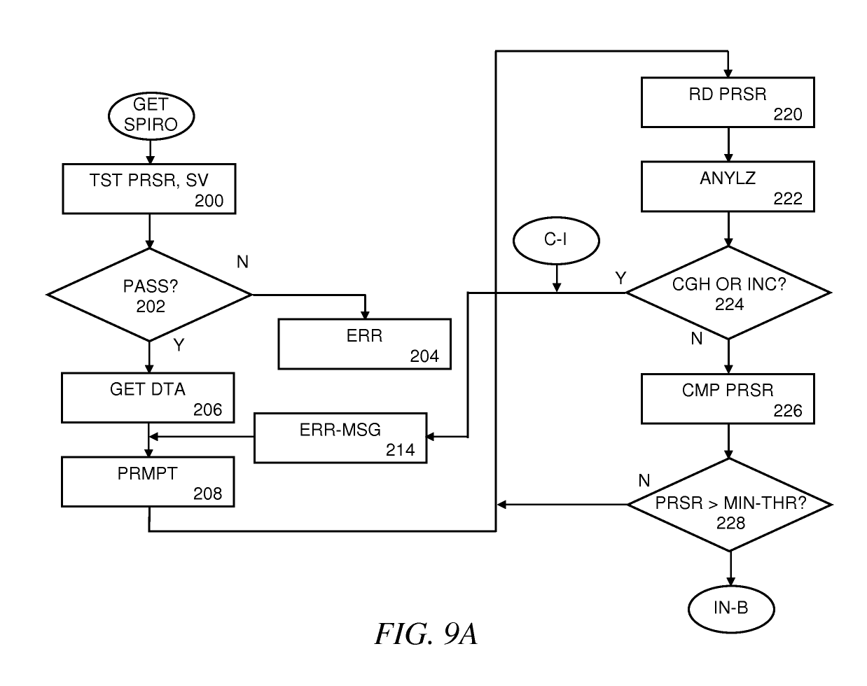

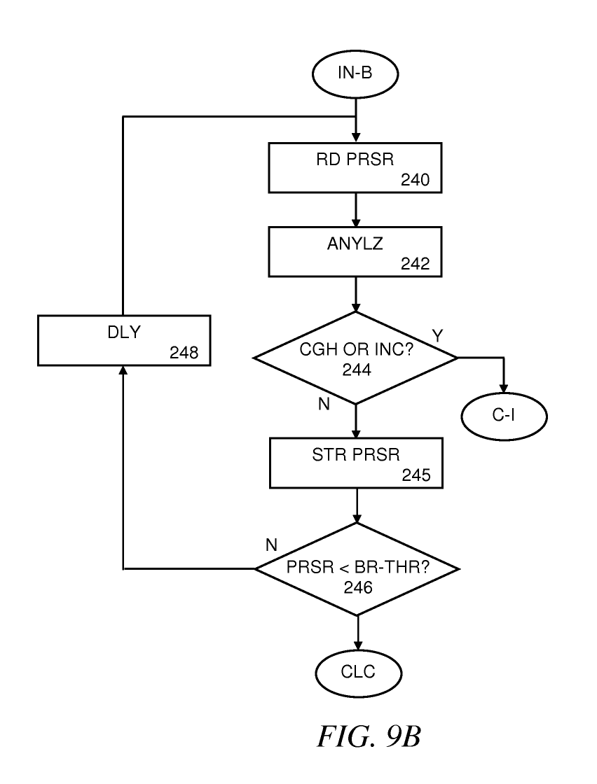

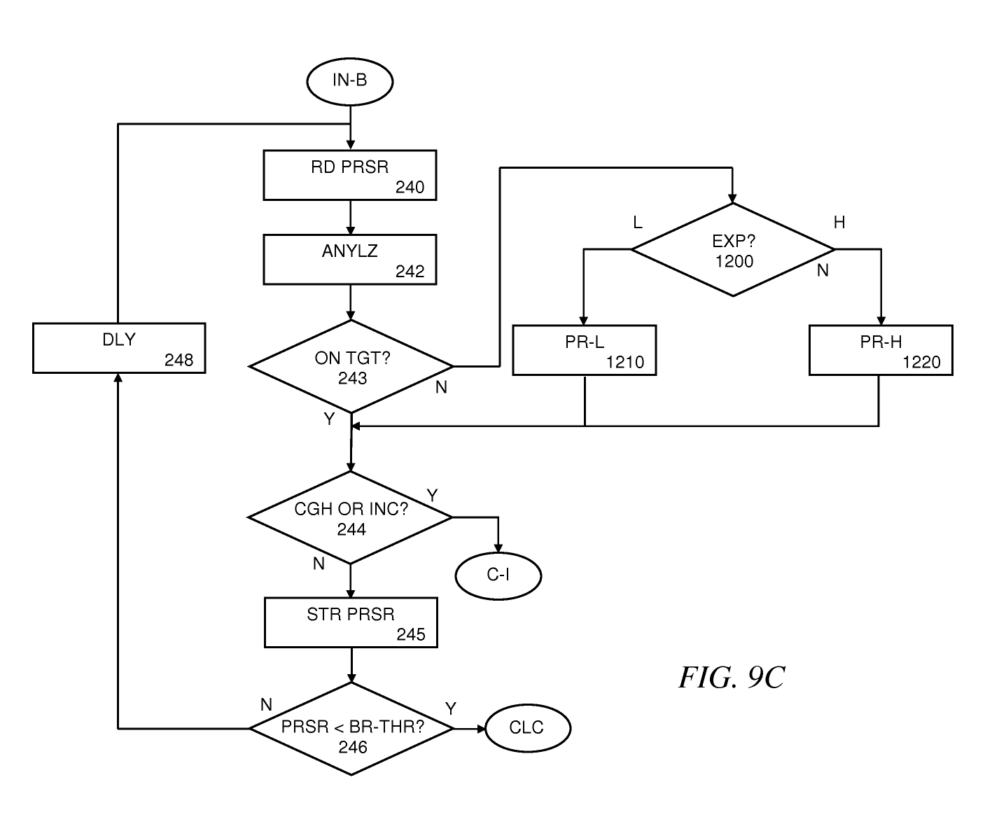

Referring to FIGS. 9A, 9B, 9C, 10 and 11, schematic views of exemplary pulmonary software flowcharts are shown. In FIG. 9A, an exemplary software flow is shown in which data is collected from the pressure sensor 20 by software running on the processor 70 of the smartphone 10. All software flows and user interfaces described here within are examples of one exemplary implementation of the pulmonary system and many other software flows and user interfaces are anticipated for achieving the same results or similar results in varying ways.

Capturing one pass of pulmonary data starts with the pressure sensor 20 being tested 200 and reading a base line atmospheric pressure. The base line atmospheric pressure is saved for later calculations. If the pressure sensor 20 is not working or in not available 202, an error message is displayed 204 (e.g. “try later” or “service required”).

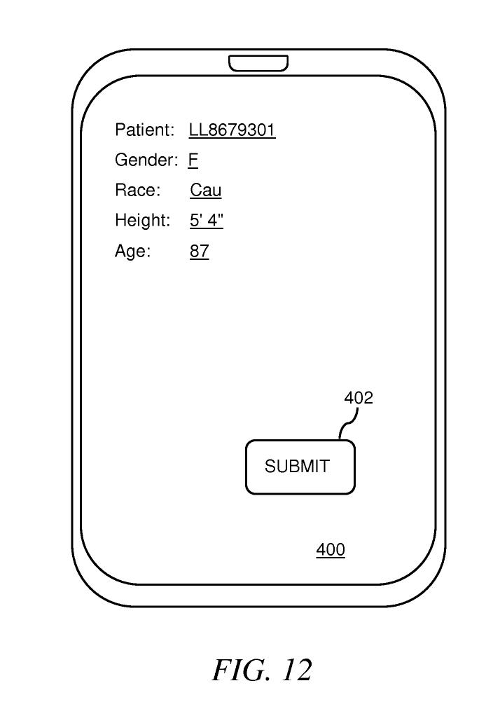

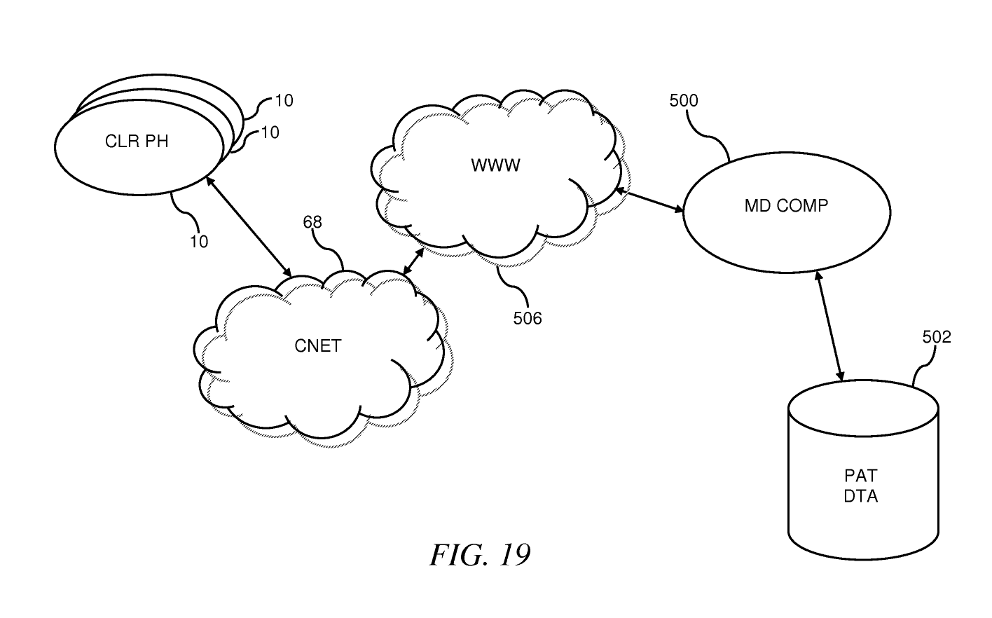

Next, data regarding the user/patient is read/captured 206. This data includes any or all parameters required to analyze and/or chart the pulmonary functions of this particular user/patient. In some embodiments, the data is entered each time the pulmonary system is used through a user interface 400 presented to the user/patient as in FIG. 12. In some embodiments, the data is entered one time through, for example, a user interface 400 presented to the user/patient as in FIG. 12 and the data is saved by the pulmonary system in the storage 74/88 of the smartphone 10, then the data is read 206. In some embodiments, data for multiple users (e.g. a husband and wife) is entered once for each user through, for example, a user interface 400 presented to the user/patient as in FIG. 12, and the data is saved by the pulmonary system in the storage 74/88 of the smartphone 10. In the later embodiment, the step of getting data 206 includes entering an identification of the current user/patient (e.g. name or number of user/patient). The data includes some or all information regarding the user/patient needed to analyze the user's/patient's pulmonary functions. In some embodiments, this data is not available at the smartphone 10 and the raw pulmonary flow/volume data is later analyzed either at the smartphone 10 or by a remote system such as a personal computer 500 (see FIG. 19). The user/patient data includes any or all of the user/patient identification (e.g. name, patient number, etc.), the age of the user/patient, the gender of the user/patient, the race of the user/patient, the height of the user/patient, etc.).

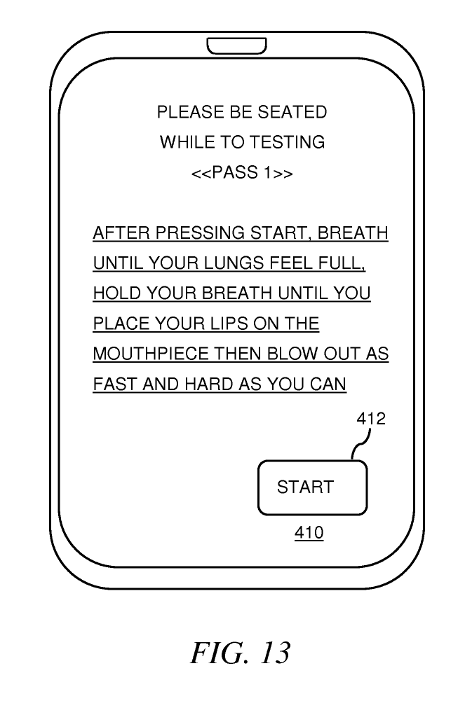

Next, the user/patient is prompted 208 as to what they need do for the test, as for example in the user interface 410 of FIG. 13. For example, the user/patient is told to press start 412 then breath in, filling their lungs as much as possible, holding that until the user/patient's lips are tightly placed on the mouthpiece 5, then the blowing out as fast and hard as possible. In some embodiments, the user/patient is informed by way of a user interface 410 while in some embodiments, the user/patient is informed by an audio message emitted on the speaker of the smartphone 10, a video presented on the display 86 of the smartphone 10, tones, vibrations, or a combination of such. Any notification/prompting is anticipated, including, but not limited to, emitting noises from the speaker, changing the display (e.g., displaying text or flashing/blinking the display), illuminating any or all of the illumination devices of the smartphone 10 (e.g., notification LEDs, camera LED), etc.

The power of encouragement during the spirometer maneuver cannot be overstressed. In some embodiments, encouragement is provided via pre-recorded audio and/or video messages are also provided during the test. For example, a video of a pre-recorded personality such as a drill sergeant or a princess, perhaps of a famous personality, is presented during the test. In another example, a pre-recorded video with audio of a doctor, nurse, or loved one (e.g. parent, child, sibling, etc.) is presented during the test. In some embodiments, pre-recorded encouragements from well-known celebrities are provided. In such, it is anticipated that a market for such pre-recorded encouragements is satisfied by pay-per-download services, etc. It is also anticipated that, for a fee, some celebrities will provide custom encouragements including the name or nickname of the user/patient. For example, a teen child might be best encouraged by their favorite baseball player or a popular singer, etc.

In another embodiment, a direct audio and/or video connection is made through the cellular network 68 or wireless network 94/96 is made and encouragement is provided directly during the test by a person at the other end of the connection. In such, the audio is presented through, for example, a speaker of the smartphone 10 and the video is displayed on the display 86 of the smartphone 10.

Although the following description focuses on measuring exhalation in the examples, there is no restriction on whether inhalation, exhalation, or gaps between the two are made, all of which are included here within.

In some embodiments, software running on the processor 70 of the smartphone 10 determines expected inhalation/exhalation capabilities of the user/patient based upon the data entered/stored, for example as in the step: data regarding the user/patient is read/captured 206. As the user performs the test, the real-time progress is compared to expected progress and, the encouragement is modified based upon this comparison. For example, a happy tone is emitted while the user is performing as would be expected and a sad tone is emitted during a period that the user falls short of expectations. Likewise, verbal or text/video cues are provided to instruct the user to adjust their breathing according to expectations, such as “breathe deeper, I know you can do it” or “you are doing just fine,” etc.

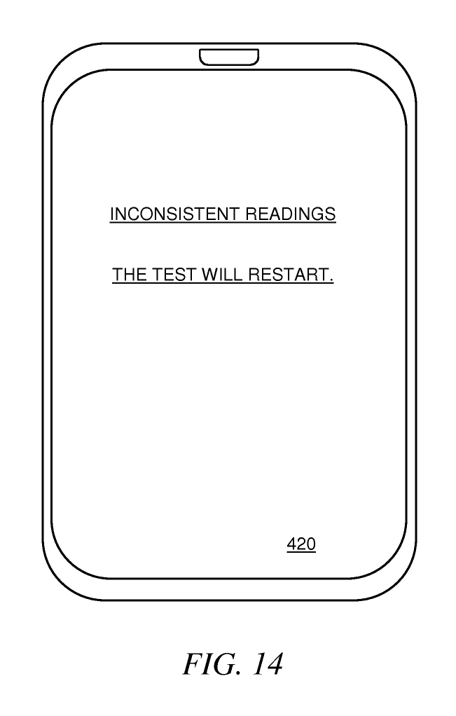

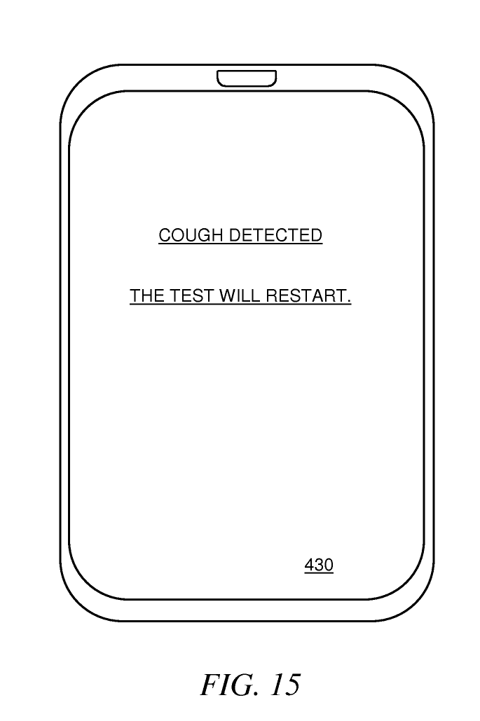

After prompting 208, a loop 220-248 is initiated in which starts with waiting for the breath to begin by reading the pressure 220 from the pressure sensor 20 and analyzing 222 the data from the pressure sensor. If the analysis indicates that the patient has coughed, the breath suddenly stopped, or the breath is incomplete 224, an error message is displayed/emitted 214 such as the error messages 420/430 of FIGS. 14 and 15 or through any audio, display, and/or illumination interface available such as a blinking LED or a voice message informing the user/patient of the issue and need to restart. The user/patient is then re-prompted 208 to restart their breadth and the loop 220-248 restarts.

If the patient hasn't coughed and the breath is not incomplete 224, the pressure that was read from the pressure sensor 20 is compared 226 to a minimum pressure threshold. For example, the minimum pressure is some percentage greater than the previously stored atmospheric pressure. In such, minor breeze or air flow fluctuations are not mistaken for the start of the user/patient breadth. If the pressure is not above the minimum pressure threshold 228, the loop 220-228 repeats waiting for the user/patient to exhale. If the pressure is above the minimum pressure threshold 228 (e.g. the user/patient has begun to exhale), the data collection loop, IN-B of either FIG. 9B or FIG. 9C, is started.

Referring to FIG. 9B, it has been determined that the patient has started to exhale into the mouthpiece 5. The loop 240-248 gathers periodic pressure readings and stores the readings in a table, array, histogram, etc. Each time through the loop 240-248, the pressure sensor 20 is read 240 and again analyzed 242, looking for irregularities such as a cough or incomplete breath. If an irregularity exists 244, the previous steps are repeated starting at prompting 214. Next the pressure is stored 245 in, for example, an array or histogram in the smartphone memory 74/88. Now, if the pressure that was read 240 is still above 246 a pre-set minimum pressure, a delay (e.g. inter-sample delay) 248 is taken before the loop 240-248 repeats. Because of user/patient specific venting in the pulmonary interface 31/31a, the pressure measurements taken from the pressure sensor 20 correlate to velocity of air per unit time (e.g. the delay time). The delay 248 is selected to provide an appropriate number of pressure samples for an expected breath cycle and any delay 248 from zero seconds and above is anticipated (e.g., no delay 248).

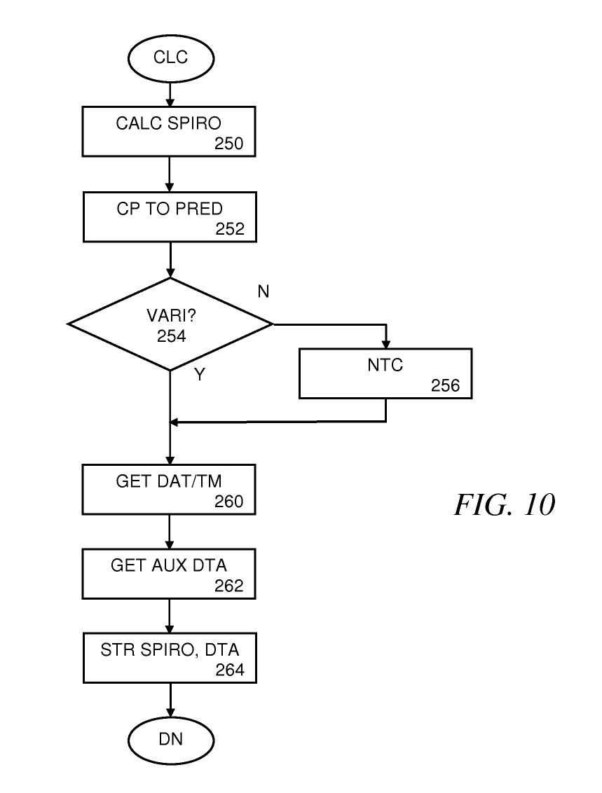

If the pressure that was read 240 is below 246 the pre-set minimum breath pressure, then it is determined that the exhalation has ended and the calculation step of FIG. 10 is performed. Note that the minimum pressure threshold and minimum breath pressure threshold are preferably selected to provide some level of hysteresis.

In some embodiments, the number of times through the loop 240-248 is counted which, multiplied by the delay, is a reading of the length of the breath. In some embodiments, the length of the breath is determined by reading the smartphone real-time clock read before and after the breath begins/ends. If the breath is too short, the user is prompted to restart starting at prompting 214, perhaps with a different message.

Referring to FIG. 9C, as with FIG. 9B, it has been determined that the patient has started to exhale into the mouthpiece 5. The loop 240-248 gathers periodic pressure readings and stores the readings in a table, array, histogram, etc. Each time through the loop 240-248, the pressure sensor 20 is read 240 and again analyzed 242.

The analysis 242 of each pressure reading 240 is reviewed 243 to determine if that breath is on target. Whether the breath is on target is determined by making various predictions of an expected breath pattern based upon the user/patient data (e.g. height, weight, etc.) as entered before the test or stored previously. If the pressure is not within a target range, one or more subsequent analysis/comparisons 1200 are made to determine the status and trajectory of the current breath 1200, such as determining if the current pressure reading is above or below an expected pressure at this point in the breath. If the pressure is, for example, higher than the expected pressure, a first prompt is provided 1220, for example, a happy tone is emitted or verbal and/or text/video cues are presented such as, “you are doing great,” etc. If the pressure is, for example, lower than the expected pressure, a second prompt is provided 1210, for example a sad tone is emitted or verbal and/or text/video cues are provided to instruct the user to adjust their breathing according to expectations, such as “breathe deeper, I know you can do it.”

In the past, a clinician or certified technician was typically required to administer the test and assure the maneuver was performed by the patient correctly. With the added capabilities of the smartphone 10, the software now monitors the user/patient performance and provides feedback as needed to encourage and assure the maneuver is performed correctly, thereby reducing or eliminating the need to perform the test in the presence of a trained caregiver. As discussed here within, it is anticipated that for some users/patients, due to lack of experience in using this system or due to other impediments, there will be times in which a caregiver (e.g. clinician, technician, physician, nurse, etc.) needs to monitor the maneuver and provide feedback to the user/patient during the maneuver. In such, the communications capabilities of the smartphone 10 are used to provide data transmission of the captured data (e.g. pressure readings, Spirograph, array of pressure readings, etc.) to the caregiver at, for example, a remote computer 500 (see FIG. 19) as well as to provide an audio and/or video feedback capability from the caregiver to the user/patient in the form of transmitted voice/video from the caregiver (e.g. from the remote computer 500, which is anticipated to be a computer 500 or another smartphone 10) to the user/patient smartphone 10.

Continuing with the flow of FIG. 9C, if an irregularity exists 244, the previous steps are repeated starting at prompting 214. Next the pressure is stored 245 in, for example, an array or histogram in the smartphone memory 74/88. Now, if the pressure that was read 240 is still above 246 a pre-set minimum pressure, a delay (e.g. inter-sample delay) 248 is taken before the loop 240-248 repeats. Because of user/patient specific venting in the pulmonary interface 31/31a, the pressure measurements taken from the pressure sensor 20 correlate to velocity of air per unit time (e.g. the delay time). The delay 248 is selected to provide an appropriate number of pressure samples for an expected breath cycle and any delay 248 from zero seconds and above is anticipated (e.g., no delay 248).

If the pressure that was read 240 is below 246 the pre-set minimum breath pressure, then it is determined that the exhalation has ended and the calculation step of FIG. 10 is performed. Note that the minimum pressure threshold and minimum breath pressure threshold are preferably selected to provide some level of hysteresis.

In some embodiments, the number of times through the loop 240-248 is counted which, multiplied by the delay, is a reading of the length of the breath. In some embodiments, the length of the breath is determined by reading the smartphone real-time clock read before and after the breath begins/ends. If the breath is too short, the user is prompted to restart starting at prompting 214, perhaps with a different message.

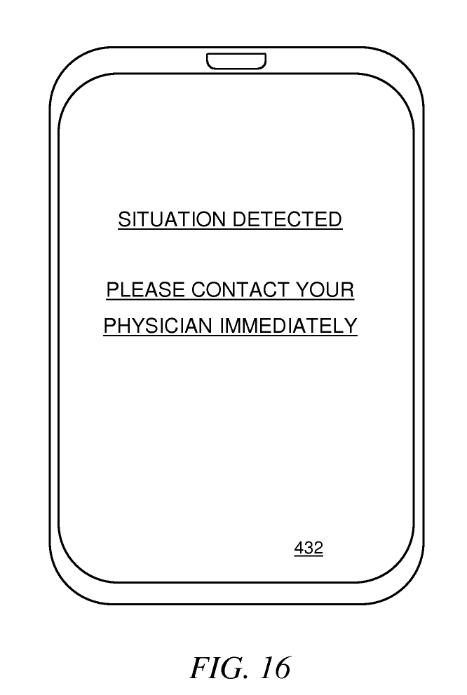

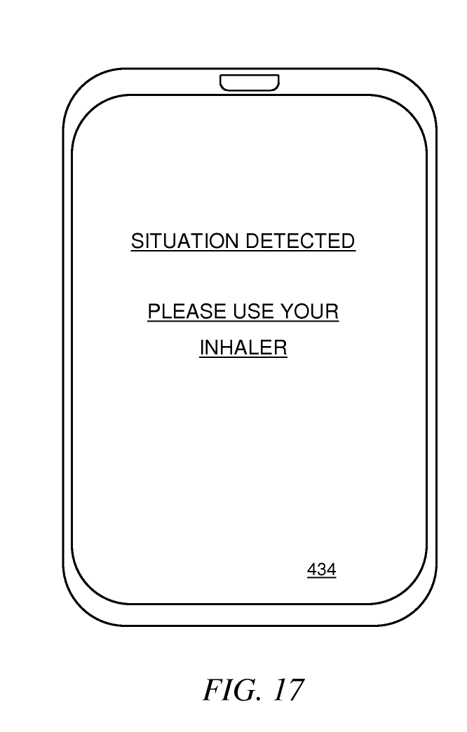

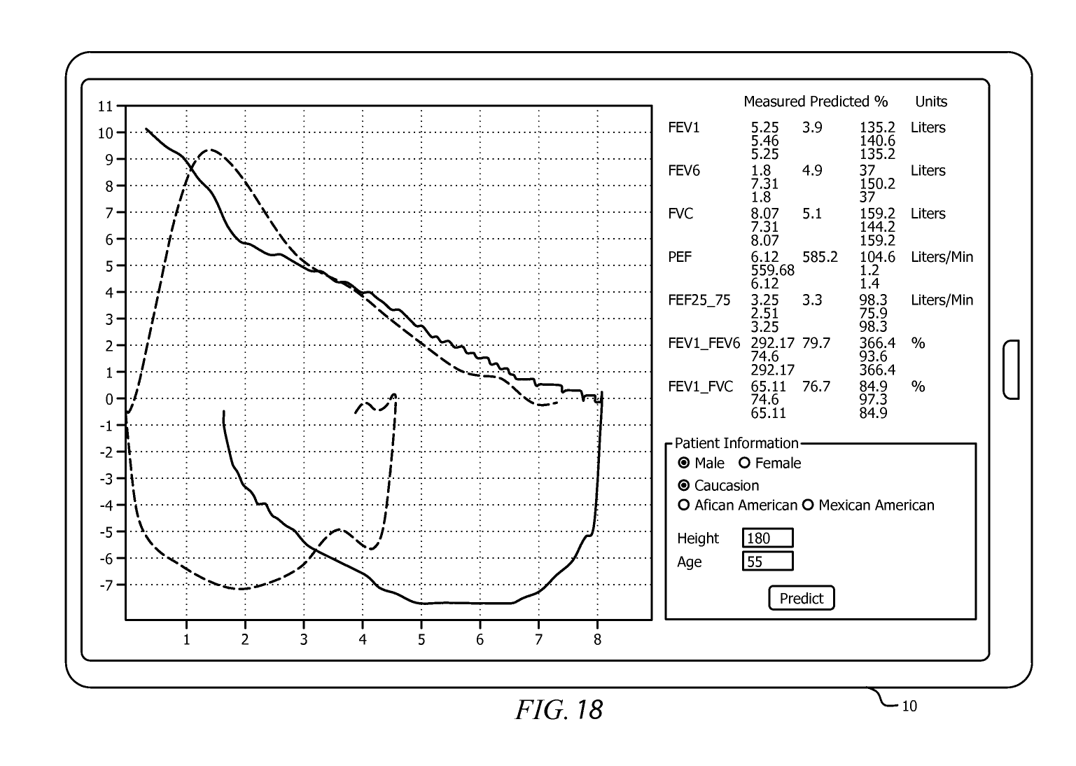

In FIG. 10, one pass of gathering and recording pulmonary data is shown. The pressure data (e.g. table, array, histogram, etc.) is processed to calculate, for example a spirogram (as shown in FIG. 18) and to generate individual readings such as FEV1, FEC, etc., (as shown in FIG. 18). The calculated values are then compared to expected or predicted values 252, such as predicted values for the user/patient based upon the user/patient data (e.g. height, weight, etc.). If the calculated values differ from the expected/predicted values by, for example, a fixed amount, a percentage amount, etc. 254, then a notice is made 256 which, in some embodiments is a message displayed on the smartphone 10 as in FIGS. 16 and 17 while in other embodiment is a message (text message, email, etc.) is sent to another person such as a caregiver, family member, physician, etc.

Now, in some embodiments, the date and/or time is accessed 260 (e.g. from the smartphone's 10 real-time clock) and any auxiliary data needed/available is also obtained 262 such as room temperature, relative humidity, etc. Some or all of the pressure data, the spirogram, the auxiliary data, and/or the user/patient data is then stored 264, for example, in the memory 74/88 of the smartphone 10.

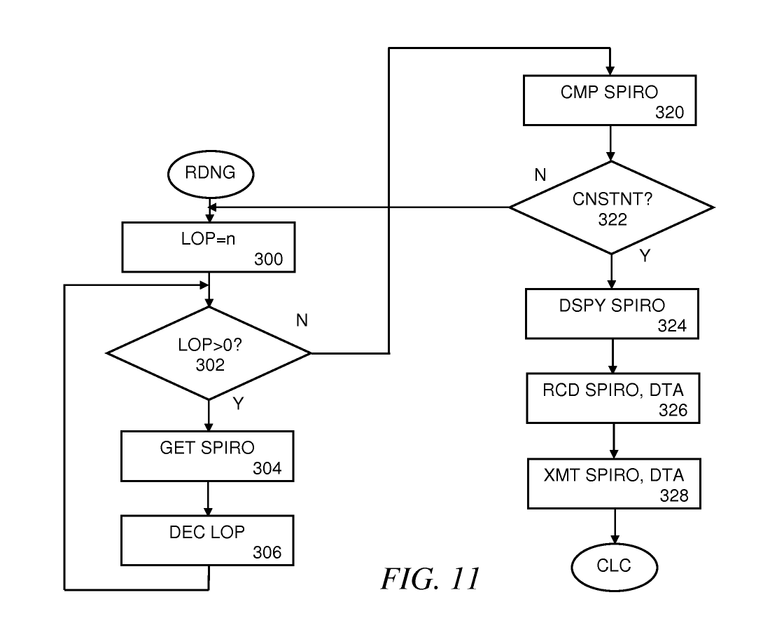

In FIG. 11, the main loop is shown for taking a complete reading. In this, a loop count is initialized 300 (any known form of looping/repeating is anticipated), then a loop 302-328 begins with checking to see if the loop count has expired (e.g. reached zero) 302. If the loop count hasn't expired 302, then one pulmonary reading (get a sprio) is performed 304 and the data is stored as described above, then the loop count is decremented (or incremented as fit) 306 and the previous steps 302-306 are repeated, capturing one set of pulmonary readings per loop. Now, the several sets of pulmonary data are compared 320 to make sure there are consistent readings. If the readings are not consistent 322, the user/patient needs to start over 300-306. If the readings are consistent 322, the final data/spirogram is displayed 324 as shown in FIG. 18 then the data/spirogram and/or auxiliary data is stored 326, in for example the smartphone memory 74/88 and, in some embodiments, the data/spirogram and/or auxiliary data is transmitted to a remote computer 500 (see FIG. 19) for analysis by other software and/or review by another (e.g. a physician).

Referring to FIGS. 12 through 17, schematic views of exemplary pulmonary data entry user interfaces are shown. In FIG. 12, the user/patient enters data specific to that user/patient into a user interface 400. The data includes any data necessary to evaluate breathing functions such as a user identification (e.g., name, patient-number, etc.), a gender (e.g. male/female), a race (e.g. Caucasian), a height (e.g. centimeters, feet/inches), an age (e.g. 87 years), etc. After the data is entered, the submit function 402 is selected to proceed and/or store the data.

In FIG. 13, a sample user interface 410 for starting the breathing test is shown. Instructions are presented along with a start function 412 which, when selected, starts the breathing test as described above.

In FIGS. 14 and 15, typical error warnings 420/430 are shown.

In FIGS. 16 and 17, typical situational warnings 432/434 are shown. These are but examples and other such warnings are anticipated. Once the breath is measured, if certain situations are detected such as obstructive or restrictive pulmonary patterns, it is determined that a situation is possible in which the user/patient needs to contact a doctor or use a prescribed inhaler. For example, if it is determined that a situation is present in which the user/patient needs to contact a doctor, a message such as the situation-detected message 432 is displayed. For example, if it is determined that a situation is present in which the user/patient needs to use their inhaler, a message is displayed such as the situation-detected message 434.

Referring to FIG. 18 illustrates a schematic view of an exemplary pulmonary data entry user interface showing an output graphic. Although several readings, calculations, and predictions are shown in FIG. 18, any pulmonary-related (or non-pulmonary related) information is anticipated to be presented in any format, including numerical format, graphical format, colors, audio, etc.

In this exemplary display, several typical pulmonary results and predictions are displayed in numerical format. For example, forced vital capacity (FVC) (a measure of lung volume and is usually reduced in diseases that cause the lungs to be smaller), forced expiratory volume in 1 second (FEV₁)) (indicate technical adequacy of the maneuver/test and help identify the anatomic location of airflow obstruction), forced expiratory flow at 25% to 75% vital capacity (FEF₂₅₋₇₅), and other calculations and predictions are shown. In some embodiments, the patient information/data is also shown to verify that the results are for a particular user/patient (e.g., one of 180 pounds and 55 years old). In some embodiments, a graphical representation of the pulmonary data is presented as shown.

FIG. 19 illustrates a schematic view of flow in an exemplary pulmonary data system. In this example, one or more smartphones 10 communicate data through the cellular network 68 and/or through a wide area network 506 (e.g. the Internet 506) to a computer 500 (e.g. a personal computer 500). The computer 500 has access to storage 502. Although any computer 500 is anticipated, in one example, the computer 500 is associated with a medical facility and/or a physician and patient information and records are stored in the storage 502. As prescribed by this physician, the patient follows the above procedures to capture pulmonary data at a frequency also prescribed by the physician. The pulmonary data is forwarded through the cellular network 58 and/or through one or more wide area networks 506 to the computer 500 associated with the medical facility and/or the physician, where the data is further analyzed and/or viewed by the physician. Although one path to/from the smartphones 10 through the cellular network 68, through the wide area network 506, and to/from the computer 500 is shown, any known data path is anticipated. For example, the Wi-Fi subsystem 96 of a smartphone 10 is used to communicate directly with the wide area network 506, which includes the Internet, and, therefore, with the computer 500.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for gathering pulmonary data, the system comprising:
a smartphone, the smartphone having a processor, a display, and a pressure sensor, the pressure sensor being operatively coupled to the processor and the display being operatively coupled to the processor; and
an interface for accepting a breath, the interface for accepting the breath having at least one channel through which the breath is fluidly interfaced between the interface for accepting the breath and the pressure sensor.

2. The system for gathering pulmonary data of claim 1, further comprising at least one vent, the vent fluidly interfaced to the interface for accepting the breath, the vent configured to release a portion of the breath to an area outside of the interface for accepting the breath.

3. The system for gathering pulmonary data of claim 2, wherein the at least one vent has an adjustment for controlling back pressure to the interface for accepting the breath.

4. The system for gathering pulmonary data of claim 3, wherein the adjustment comprises a device for occluding one or more of the at least one vents.

5. The system for gathering pulmonary data of claim 1, further comprising software, the software running on the processor reads data from the pressure sensor, the software analyzes the data to generate pulmonary readings, and the software displays the pulmonary readings on the display.

6. The system for gathering pulmonary data of claim 1, further comprising software, the software running on the processor reads data from the pressure sensor and the software analyzed the data to generate a pulmonary Spirograph, the software displays the pulmonary Spirograph on the display.

7. The system for gathering pulmonary data of claim 1, wherein the smartphone further comprises means for sending data and the system for gathering pulmonary data further comprising software, the software running on the processor reads data from the pressure sensor, the software analyzes the data to generate pulmonary readings, and the software sends the pulmonary readings to a remote computer through the means for sending data.

8. The system for gathering pulmonary data of claim 1, wherein the smartphone further comprises means for sending and the system for gathering pulmonary data further comprising software, the software running on the processor reads data from the pressure sensor, the software sends the data to a remote computer through the means for sending data.

9. The system for gathering pulmonary data of claim 8, wherein the software further sends a date and a time of when the software read the data, an atmospheric pressure reading at the time, a temperature measurement at the time, a patient identification with the data to the remote computer through the means for sending data.

10. A method of gathering pulmonary data comprising:
affixing a custom back cover to a smartphone, the custom back cover comprising an interface for accepting a breath from a user and the custom back cover having at least one channel through which the breath is fluidly interfaced between the interface for accepting the breath and a pressure sensor of the smartphone, the pressure sensor operatively coupled to a processor of the smartphone;
starting an application, the application running on the processor of the smartphone;
the application displaying instructions on a display of the smartphone, the display operatively coupled to the processor;
a user following the instructions, the user exhaling into the interface for accepting a breath; and
the application gathering exhalation data from the pressure sensor while the user is exhaling.

11. The method of claim 10, wherein the step of gathering the exhalation data further comprises analyzing the exhalation data while the user is exhaling and providing feedback to the user regarding how well the user is exhaling as expected by the application.

12. The method of claim 10, further comprising a step of analyzing the exhalation data after the step of gathering the exhalation data, then comparing results of the analyzing to previous results.

13. The method of claim 10, further comprising a step of generating a Spirograph from the exhalation data after the step of gathering the exhalation data.

14. The method of claim 10, further comprising a step of reading data regarding the user and sending the data regarding the user and the exhalation data to a remote computer.

15. The method of claim 10, wherein the step of gathering the exhalation data further includes emitting tones from a sound producing element of the smartphone until the user exhaling abates.

16. An apparatus for measuring pulmonary capabilities, the apparatus comprising:
a pulmonary interface, the pulmonary interface having an interface for accepting a breath and the pulmonary interface having at least one channel through which at least a portion of the breath is fluidly interfaced between the interface for accepting a breath and a pressure sensor of a smartphone.

17. The apparatus of claim 16, further comprising at least one vent in the pulmonary interface, each of the at least one vent in fluid communication with the breath interface and with the channel.

18. The apparatus of claim 17, wherein the vents are adjustable in air volume.

19. The apparatus of claim 16, wherein the breath interface accepts a sampling tube around which a user places lips of the user while exhaling.

20. The apparatus of claim 16, wherein the breath interface is fluidly interfaced to the pressure sensor of the smartphone through an elongated sampling tube extension, thereby enabling use of the breath interface while viewing a display of the smartphone.
